(19) United States
(12) Patent Application Publication
(10) Pub. No.: US 2014/0340634 A1
(43) Pub. Date: Nov. 20, 2014

(54) OPTICAL COHERENCE TOMOGRAPHY SYSTEMS AND METHODS

(71) Applicant: Wasatch Photonics, Inc., Logan, UT (US)

(72) Inventors: Roman V. Kuranov, Cary, NC (US); Scott M. Norton, Durham, NC (US); William J. Brown, Durham, NC (US)

(21) Appl. No.: 14/246,094

(22) Filed: Apr. 5, 2014

Related U.S. Application Data
(60) Provisional application No. 61/809,285, filed on Apr. 5, 2013.

(51) Int. Cl. A61B 3/10; A61B 3/14

(52) U.S. Cl. CPC A61B 3/102; A61B 3/14
USPC 351/206

(57) ABSTRACT

An optical coherence tomography system may have a reflector system that reflects light from a light source to provide a plurality of light rays that fall within different wavelength ranges, optical componentry that directs a subset of a first beam of light including the plurality of light rays to an object to be imaged, a detector that detects the subset after reflection from the object, and signal processing componentry that processes signals from the detector to provide an optical coherence tomography image of the object. The reflector system may be configured such that the optical pathways followed by the light rays change in length at an even increment, so that the first beam of light will project wavelength groups at relatively even time increments. The optical pathways may be collinear or offset from each other. An interleaver may be used to enable detection of the first beam by multiple detectors.

### RELATED APPLICATIONS

PCT Application No. PCT/US2013/057534, filed Aug. 30, 2013 and entitled METHOD AND APPARATUS FOR ULTRAFAST MULTI-WAVELENGTH PHOTOTHERMAL OPTICAL COHERENCE TOMOGRAPHY is incorporated herein by reference, as though set forth herein in its entirety.

### TECHNICAL FIELD

The present invention relates to imaging techniques, and more specifically, to optical coherence tomography systems and methods that provide enhanced data acquisition and/or display rates.

### BACKGROUND

Optical Coherence Tomography (OCT) is a non-invasive imaging tool providing different types of 2-D and 3-D microscopic information about biological and other objects. Through the use of interferometry with relatively long wavelength light (typically near infrared), the light may penetrate some distance into the medium to be imaged, thereby providing details regarding subsurface morphology in the resulting image. OCT is a routinely used tool in ophthalmology and is also being commercialized for use in other health care fields such as cardiology and dentistry. OCT tools are also being developed for industrial applications including 3-D memory and coating quality.

One of the major limiting factors of OCT applications is the acquisition rate of the one dimensional (1-D) in-depth profiles or “A-scan data” and the corresponding display rate of the OCT data. The OCT data can be displayed in two-dimensional (2-D) “B-scan” or in three-dimensional (3-D) “C-scan” formats. The B-scans may consist of consecutively-numbered A-scans (usually 256, 512 or 1024 or any other number of A-scans). C-scans may include consecutively-numbered B-scans (128, 256 and 1024 are typical values) projected in a variety of ways on the 2-D screen. The acquisition rates of the commercially available OCT instruments are generally limited to 100 kA-scans/s to 200 kA-scans/s. Acquisition rates of Mega A-scans/s and faster (“Megahertz OCT”) may eliminate motion artifacts, significantly increase the investigated area, and allow acquisition of valuable functional and dynamic information, thus making OCT instruments more valuable for applications in which it is currently used, and broadening the use of OCT technologies to exciting new applications.

Megahertz OCT may be particularly advantageous for the following applications:
Ophthalmic Imaging—OCT imaging is limited by the motion of the patient relative to the system. Wide-field retinal OCT may benefit from high speed OCT because more rapid OCT scanning may enable imaging of a large percentage of the retina in less than the ⅓ of second response time of the blink reflex. Functional OCT may be limited by multimodal micro-movements of the eye; Megahertz OCT may provide better functional imaging, for example, to visualize blood oxygenation and nerve firing in the retina.
Intravascular Imaging—since OCT generally does not penetrate blood, current OCT imaging techniques may be limited by the amount of clearing agent injected into the blood stream. Thus, imaging time may be limited to a time window of approximately 4-6 seconds; the time window may, in some cases, be up to 10 seconds. High speed OCT may allow coverage of a large area or more dense coverage within a given area. MHz-OCT may open the door to imaging that covers nearly 100% of blood vessels adding up to 20-100 millimeters or longer.
Other Imaging—applications requiring imaging of a large area, such as OCT imaging of the GI tract, may, with current technology, require multiple A-scans at a given location or A-scans with minimal relative motion of the sample. Such applications may benefit from Megahertz OCT because the imaged area may be enlarged and/or more motion of the sample may be permissible without adversely affecting the accuracy of the image.

Several methods have been proposed to achieve higher A-scan acquisition rates in optical coherence tomography. Such methods are generally limited by a number of factors including, but not limited to, (1) cost and/or stability problems associated with specialized components such as FDML lasers, (2) difficulties synchronizing multiple OCT signals, (3) light loss, resulting in degradation of the quality of the OCT image, and (4) low signal-to-noise ratios, again resulting in degradation of the OCT image quality. It would be advantageous to provide systems and methods of increasing the A-scan acquisition rates that remedy the shortcomings of the prior art.

### SUMMARY

An optical coherence tomography system according to the invention may provide faster A-scan acquisition rates that solve many of the shortcomings of the prior art. In one embodiment, the optical coherence tomography system may have a reflector system, optical componentry in the form of an optical splitter or splitters that divide light into an object portion and a reference portion, optical componentry that directs the object portion of a first beam of light including the plurality of light rays to an object to be imaged, optical componentry that directs the reference portion of a second beam of light including the plurality of light rays to a reference for the first beam path, optical componentry in the form of an optical combiner or combiners that combine the reference and object portions to produce the OCT signal via interference, a detector that detects the subset after reflection from the object, and signal processing componentry that processes signals from the detector to provide an optical coherence tomography image of the object. The reference path and object path may significantly overlap except at the very distal end to provide the “common-path geometry” of the OCT system.

The reflector system may have distributed reflectors designed to provide a plurality of light rays that fall within different wavelength ranges. The reflectors of the reflector system may be offset from each other such that the optical pathways followed by the light rays change incrementally, thereby providing a constant delay between adjacent light rays. Some optical tracts can exhibit significant dispersion due to the fact that the light at different wavelengths may propagate at different velocities. The dispersion can be compensated for by appropriate offset correction between the reflectors, thus providing a constant delay between adjacent light rays. Alternatively or additionally, non-constant delays between adjacent light rays can be compensated for in software by acquiring information related to the non-linearity values, for example, via an additional reference interferometer. The light rays may converge at a convergence location from which the light rays, combined, define the first beam of light.

The optical pathways may be collinear, and the reflectors may each be designed to reflect only the desired wavelength range of light. Alternatively, a dispersive element may be used to disperse the light from the light source along optical pathways that are offset from each other; each reflector may then reflect substantially all incident light to the convergence location.

If desired, one or more optical amplifiers may be used to amplify the light from the light source, the light of the light rays, and/or the first beam of light. The light source may advantageously be a pulsed broadband supercontinuum laser, which may have an ultra-wide bandwidth that facilitates ultra-high resolution OCT imaging. Another pulsed broadband source that may be advantageously used in connection with the present invention is a femtosecond laser.

An A-scan trigger may be positioned to receive the light from the light source, the light of the light rays, and/or the first beam of light and to emit, in response, an a-scan trigger pulse that can be used to initiate detection of the A-scan data that will be embodied in the light rays of the first beam after reflection from the object to be imaged. The A-scan trigger pulse may be differentiated from the A-scan data by amplitude and/or wavelength. If desired, a wavelength division multiplexer (WDM) may be used to multiplex the A-scan trigger pulse into a pathway followed by the first beam.

The signal processing componentry may include one or more analog-to-digital converters. Advantageously, the wavelength-varied pulsing nature of the first beam may be used to clock the ADC, thereby obviating the need for a separate clocking mechanism.

If desired, multiple reflector systems may be used. The light from the light source may be divided by a coupler or other element into multiple components, each of which is transmitted to a reflector system. The light from the reflector systems may, if desired, be combined into the first beam.

In one embodiment, the optical coherence tomography system may include an interleaver that receives the first beam and divides the first beam into multiple portions that can be detected by multiple detectors. Multiple ADC's may also be used. Such interleaving may reduce the required detection rate of the detectors and/or reduce the required digitization rate of the ADC's.

The signal processing componentry may advantageously be designed to process the A-scans without the need for phase stabilization; this may be facilitated by the relative predictability in k-numbers of the light rays. If desired, non-sequential k-numbers may be used to facilitate interleaving with a component such as an interleaver, as described previously, and/or performing compressed OCT sensing.

The reflector system may include a fiber Bragg grating (FBG) array. The fiber of such an FBG array may advantageously be chirped along its length. A large number of reflectors may be provided in such an FBG array to facilitate ultra-high resolution OCT imaging and/or ultra-deep OCT imaging.

The relatively high A-scan acquisition rate provided by OCT systems according to the invention may enable their use for a wide variety applications for which conventional OCT systems are ill-suited due to insufficient A-scan acquisition rates. In some embodiments, an OCT system with distributed reflectors according to the invention may be combined with one or more other ultrafast imaging systems. The two systems may share one or more components, such as the signal processing componentry. The combined imaging techniques may yield additional information about one or more objects to be imaged.

### BRIEF DESCRIPTION OF THE DRAWINGS

FIG. 1 is a schematic diagram illustrating an optical coherence tomography system according to one embodiment of the invention.

FIGS. 2A and 2B are schematic diagrams illustrating illumination components featuring two configurations of the distributed reflectors according to various embodiments of the invention.

FIGS. 3A, 3B, and 3C are schematic diagrams illustrating three different illumination components that include optical amplification.

FIG. 4 is a chart that illustrates the exemplary formation of optical output from an illumination component such as the illumination component of FIG. 1.

FIGS. 5A, 5B, and 5C are schematic diagrams illustrating various illumination components that provide exemplary schema for combining optical and/or data pathways for A-scan pulses and A-scan data.

FIG. 6 is a schematic view illustrating an optical coherence tomography system in which the A-scan trigger pulse is separated from the A-scan data through the use of a wavelength division multiplexer in the detector pathway of the OCT system.

FIG. 7 is a chart that illustrates the exemplary formation of optical output from an OCT system with commercially available components.

FIG. 8 is a table illustrating the parameters of major components that may be used to implement a distributed reflector OCT system.

FIG. 9 is a schematic view illustrating an illumination component according to another alternative embodiment of the invention.

FIG. 10 is a chart that illustrates the exemplary formation of optical output from an OCT system including the illumination component of FIG. 9.

FIG. 11 is a schematic view illustrating an illumination component according to another alternative embodiment of the invention.

FIG. 12 is a schematic diagram illustrating an optical coherence tomography system in which interleaving of the A-scan data is used to expedite analog-to-digital conversion.

FIG. 13 is a schematic view illustrating an interleaver as in FIG. 12.

### DETAILED DESCRIPTION

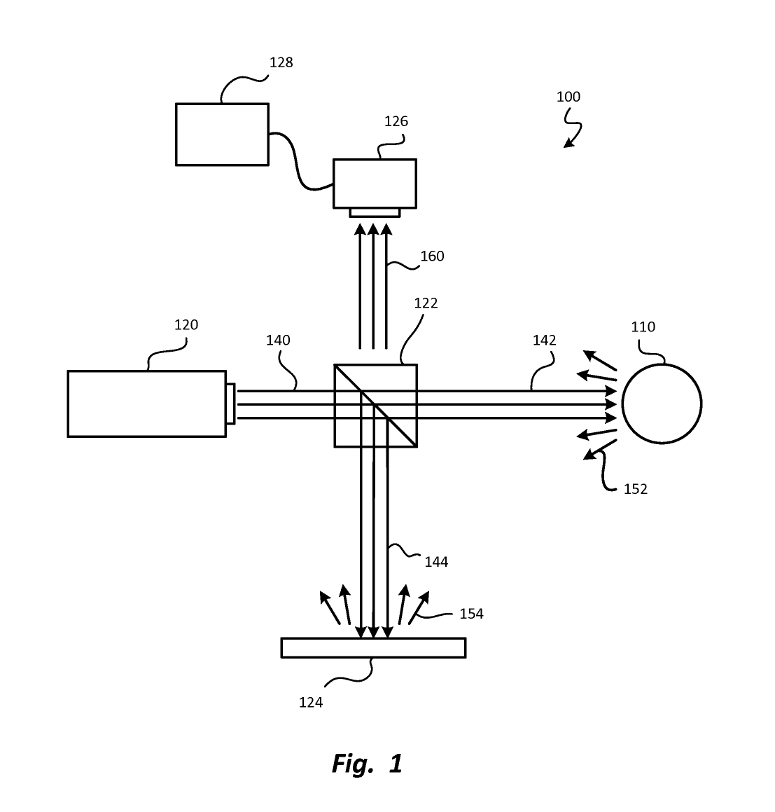

Various embodiments of the invention will now be described in greater detail in connection with FIGS. 1-13. The drawings and associated descriptions are merely exemplary; the scope of the invention is defined not by these, but by the appended claims.

Referring to FIG. 1, a schematic diagram illustrates an optical coherence tomography system, or system 100 according to one embodiment of the invention. The system 100 may be designed to record an OCT image of an object 110, which may be an article of manufacture, a part of the body (such as a human eye), or any other object to be imaged.

The system 100 may have a wide variety of configurations. According to the embodiment shown, the system 100 may include an illumination component 120, a splitter 122, a reference reflector 124, a detector 126, and signal processing componentry 128. The splitter 122 and the reference reflector 124 may be constituents of optical componentry of the system 100, which optical componentry may be designed to direct light from the illumination component 120 at the object 110 and at the detector 126.

The illumination component 120 may be any of a wide variety of light sources known in the art of OCT imaging. In certain embodiments, the illumination component 120 may be a broadband pulsed light source, which may emit pulses of light in a range of frequencies and/or wavelengths. The wavelengths may be selected to permit some penetration of the surface of the object 110 to be imaged. If desired, the wavelengths may generally be at or near the infrared portion of the electromagnetic spectrum to facilitate such penetration of objects such as tissue samples. In this application, “light” is not limited to visible light, but may include electromagnetic radiation of any frequency or wavelength.

The illumination component 120 may be configured to project light in a plurality of wavelength groups that are generally distinct (i.e., generally non-overlapping or overlapping only in part) with each other. The wavelength groups may be spatially separated such that, at any given time, the illumination component 120 is generally emitting light with wavelengths within only one of the wavelength groups. Such emission may facilitate more rapid A-scan acquisition because each wavelength group may be individually scanned in sequence. Various embodiments of illumination components 120 according to the invention will be shown and described hereafter.

The illumination component 120 may project a first beam 140 of light toward the splitter 122. The first beam 140 may be a broadband pulsed beam with discrete wavelength groupings as described above. The splitter 122 may be designed to receive the first beam 140 and divide the first beam 140 into two components: an object beam 142 and a reference beam 144. The splitter 122 may have any configuration known in the art. If desired, the splitter 122 may have the shape of a rectangular prism, which may include two triangular prisms as shown.

A portion of the first beam 140 may pass directly through the splitter 122 to define the object beam 142, and the remainder of the first beam 140 may reflect from the interface between the prisms to define the reference beam 144. The object beam 142 and the reference beam 144 are shown displaced by an angle of 90°, but may be displaced by a variety of different angles in different embodiments. If desired, the positions of the object 110 and the reference reflector 124 may be swapped so that the object beam 142 is reflected within the splitter 122 while the reference beam 144 passes directly through the splitter. In some embodiments, the splitter 122 may provide approximately 50% reflectance so that approximately half the light of the first beam 140 is conveyed to the object beam 142 and the other half is conveyed to the reference beam 144.

The object beam 142 may project toward the object 110, which may reflect the light from the object beam 142 back toward the splitter 122 in the form of a reflected object beam 152. Similarly, the reference beam 144 may project toward the reference reflector 124, which may redirect the reference beam 144 back toward the splitter 122 as a reflected reference beam 154. At least a portion of the reflected object beam 152 may be redirected by the splitter 122 toward the detector 126, while at least a portion of the reflected reference beam 154 may pass through the splitter 122. The portion of the reflected object beam 152 may combine with the portion of the reflected reference beam 154 to define an imaged beam 160.

The imaged beam 160 may exhibit an interference pattern between its component portions of the reflected object beam 152 and the reflected reference beam 154. This interference pattern may be recorded by the detector 126 and may define the image. Notably, the object beam 142 may reflect from subsurface features within the object 110 to enable the detector 126 to non-invasively record subsurface morphology of the object 110. The detector 126 may include the components necessary to image the individual wavelength groupings emitted by the illumination component 120, thereby providing an enhanced A-scan rate.

The signal processing componentry 128 may receive signals from the detector 126 and may process the signals to provide the desired OCT image of the object 110. In some embodiments, the signal processing componentry 128 may include a computing device such as a personal computer. Additionally or alternatively, the signal processing componentry 128 may include various stand-alone, function-specific components. The signal processing componentry 128 may include analog-to-digital converters (ADC's), digital signal processors, interleavers, and/or a variety of other components known in the art. Thus, the signal processing componentry may perform a number of tasks including but not limited to analog-to-digital conversion, compilation of image data, and/or output of the resulting image to a display screen or recording medium.

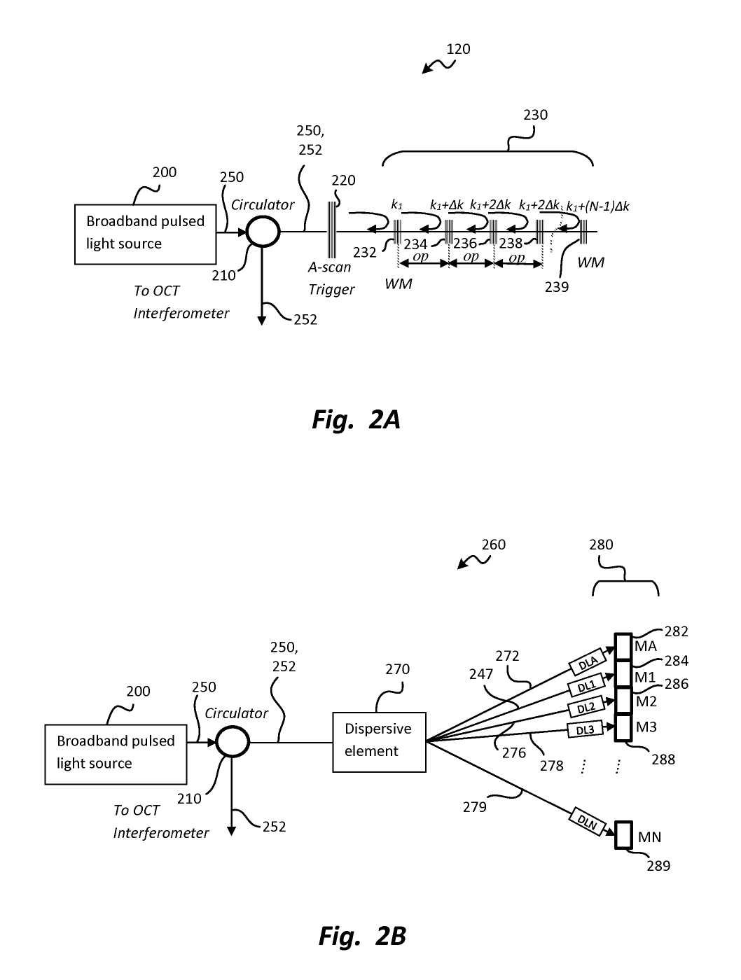

Referring to FIGS. 2A and 2B, schematic diagrams illustrate illumination components according to various embodiments of the invention. More precisely, FIG. 2A illustrates the illumination component 120 of FIG. 1, and FIG. 2B illustrates an illumination component 260 according to one alternative embodiment of the invention.

FIG. 2A illustrates a relatively simple configuration that provides the wavelength grouping described in connection with FIG. 1 through the use of a plurality of distributed deflectors. More precisely, the illumination component 120 of FIG. 2A may have a light source 200 that generates pulses of broadband light. Further, the illumination component 120 may have a circulator 210 that redirects the light to and from other modules of the illumination component 120 according to a rotation or other pattern, an A-scan trigger 220 that signals another component of the system 100, such as the detector 126 and/or the signal processing componentry 128, to initiate an A-scan, and a reflector system 230 that reflects light from the light source 200 in a manner that provides the desired wavelength groups.

The reflector system 230 may have a series of reflectors, which may each be tuned to reflect light within a certain wavelength range. As shown in FIG. 2A, the reflector system 230 may include a first reflector 232, a second reflector 234, a third reflector 236, a fourth reflector 238, and an nth reflector 239. The space between the fourth reflector 238 and the nth reflector 239 may be occupied by one or more additional reflectors (not shown), if desired, so that the reflector system 230 has more than five reflectors. Alternatively, one or more of the first reflector 232 through the fourth reflector 238 may be omitted so that the reflector system 230 has fewer than five reflectors. The reflectors of the reflector system 230 may generally be arranged along the pathway of the light exiting the circulator 210 from the light source 200.

The first reflector 232, the second reflector 234, the third reflector 236, and the fourth reflector 238 of the reflector system 230 may each be tuned to reflect light within a predetermined wavelength range (a wavelength group), and to permit pass-through of light outside the wavelength group. Thus, the first reflector 232 may reflect only light within a first wavelength group back toward the circulator 210, while allowing light with a wavelength above or below the predetermined wavelength range of the first wavelength group to pass through to the second reflector 234. Similarly, the second reflector 234, the third reflector 236, and the fourth reflector 238 may reflect light within second, third, and fourth wavelength ranges, respectively, back to the circulator 210, while allowing higher or lower wavelengths to pass through. The nth reflector 239 may, if desired, be designed to reflect all remaining wavelengths back to the circulator 210.

The reflectors of the reflector system 230 may be spaced apart according to a consistent increment. More specifically, the reflectors of the reflector system 230 may be spaced apart by an even increment “op” such that the wave numbers k₁ through kₙ (i.e., optical frequencies) of the wavelength groups are also evenly spaced apart, such as by the increment Δk. This will be discussed in greater detail subsequently.

In operation, the light source 200 may emit pulsed broadband light 250, which may be directed by the circulator 210 to the A-scan trigger 220 and the reflector system 230. The pulsed broadband light 250 may pass through the A-scan trigger 220 and may then be reflected from the corresponding reflector of the reflector system 230. The difference in optical pathway lengths between the reflectors of the reflector system 230 may divide reflectance of the wavelength groups so that the reflector system 230 provides pulsed, wavelength-grouped light 252 back to the circulator 210. The circulator 210 may emit the pulsed, wavelength-grouped light 252 back toward the remainder of the system 100, such as toward the splitter 122.

The A-scan trigger 220 may trigger scanning upon passage of light with predetermined characteristics, such as amplitude or wavelength, into the reflector system, through the A-scan trigger 220. This may occur as the light passes from the circulator 210 to the reflector system 230 and/or as the light passes from the reflector system 230 to the circulator 210.

FIG. 2B illustrates an illumination component 260 that may accomplish wavelength grouping in a somewhat different manner. Like the illumination component 120, the illumination component 260 may have light source 200 and a circulator 210. The illumination component 260 may also have an A-scan trigger (not shown), which may be located at any of a variety of locations. If desired, the A-scan trigger may be adjacent to the circulator 210 as in FIG. 2A.

In place of the reflector system 230, the illumination component 260 may have a dispersive element 270 and a reflector system 280. The dispersive element 270 may divide the light from the light source 200 by wavelength, and emits light of each wavelength group along a different optical pathway, thereby spatially separating each wavelength group. For example, the dispersive element 270 may emit light of a first wavelength group (a “first light ray”) along a first optical pathway 272, emit light of a second wavelength group (a “second light ray”) along a second optical pathway 274, emit light of a third wavelength group (a “third light ray”) along a third optical pathway 276, emit light of a fourth wavelength group (a “fourth light ray”) along a fourth optical pathway 278, and emit light of an nth wavelength group (an “nth light ray”) along an nth optical pathway 279. The dispersive element 270 may divide the broadband light 250 from the light source 200 into more or fewer than five wavelength groups.

The dispersive element 270 may be a diffractive grating, combination of prisms, or the like. The dispersive element 270 may optionally be functionally combined with the delay lines DLA, DL1, DL2, DL3, and DLN in FIG. 2B. For this purpose, for example, arrayed waveguide gratings can be used.

The reflector system 280 may include a first reflector 282, a second reflector 284, a third reflector 286, a fourth reflector 288, and an nth reflector 289, which may be indicated by the symbols MA, M1, M2, M3, and MN in FIG. 2B. Each reflector of the reflector system 280 may be positioned in the corresponding optical pathway of light emitted by the dispersive element 270. Thus, the first reflector 282 may be positioned to reflect light from the first optical pathway 272, the second reflector 284 may be positioned to reflect light from the second optical pathway 274, the third reflector 286 may be positioned to reflect light from the third optical pathway 276 the fourth reflector 288 may be positioned to reflect light from the fourth optical pathway 278, and the nth reflector 289 may be positioned to reflect light from the nth optical pathway 279. The first reflector 282, the second reflector 284, the third reflector 286, the fourth reflector 288, and the nth reflector 289 need not permit any portion of the light to pass through; accordingly, they may be designed for 100% reflectance.

The reflectors of the reflector system 280 may be positioned and/or oriented to reflect the light to a single convergence location, such as the end of a fiber optic cable or other medium. In this application, “converge” of optical pathways relates to optical pathways that intersect at a single convergence location so that light travelling along each of the optical pathways travels through the convergence location. The optical pathways may be located at angles to each other, or may be collinear. Optical pathways that converge need not do so in a manner that causes light travelling along the optical pathways to reach the convergence location simultaneously; indeed, it may be advantageous to vary the length of optical pathways before they converge so that light from the optical pathways arrives at the convergence location at a plurality of different times.

If desired, the first reflector 282, the second reflector 284, the third reflector 286, the fourth reflector 288, and the nth reflector 289 may be positioned to reflect the light back along the first optical pathway 272, the second optical pathway 274, the third optical pathway 276, the fourth optical pathway 278, and the nth optical pathway 279, respectively, to the dispersive element 270. The dispersive element 270 may also be configured to receive and multiplex the light from the reflector system 280 for conveyance back to the circulator 210 as wavelength-grouped light 252.

Alternatively, the various wavelength groups may be reflected by a single reflector (not shown in the FIG. 2B) that is long enough to cover the wavenumbers/wavelengths of interest. In either case, additional optical elements such as lenses may be used to increase effectiveness of the light coupling back the single convergence location. For example, in the case of a single long reflector, refractive optics to match the wave front of the light with the reflector surface can be used. In case of the multiple reflectors, such as the first reflector 282, the second reflector 284, the third reflector 286, the fourth reflector 288, and the nth reflector 289, each reflector's angle can be individually adjusted for high return light efficiency.

Once the wavelength-grouped light 252 has been obtained from the combination of the first, second, third, fourth, and nth light rays, the wavelength-grouped light 252 may be conveyed back to the circulator 210. The circulator 210 may emit the pulsed, wavelength-grouped light 252 back toward the remainder of the system 100, such as toward the splitter 122.

If desired, the optical pathways followed by the first reflector 282, the second reflector 284, the third reflector 286, the fourth reflector 288, and the nth reflector 289 may also differ in length to provide a time delay between the emission of each wavelength group. If desired, the optical pathways between the first reflector 282, the second reflector 284, the third reflector 286, the fourth reflector 288, and the nth reflector 289 may be staggered along a common increment to provide a relatively consistent delay between emission of each wavelength group and the following wavelength group. Thus, the first optical pathway 272, the second optical pathway 274, the third optical pathway 276, the fourth optical pathway 278, and the nth optical pathway 279 may be represented by delay lines indicated by the symbols DLA, DL1, DL2, DL3, and DLN, respectively.

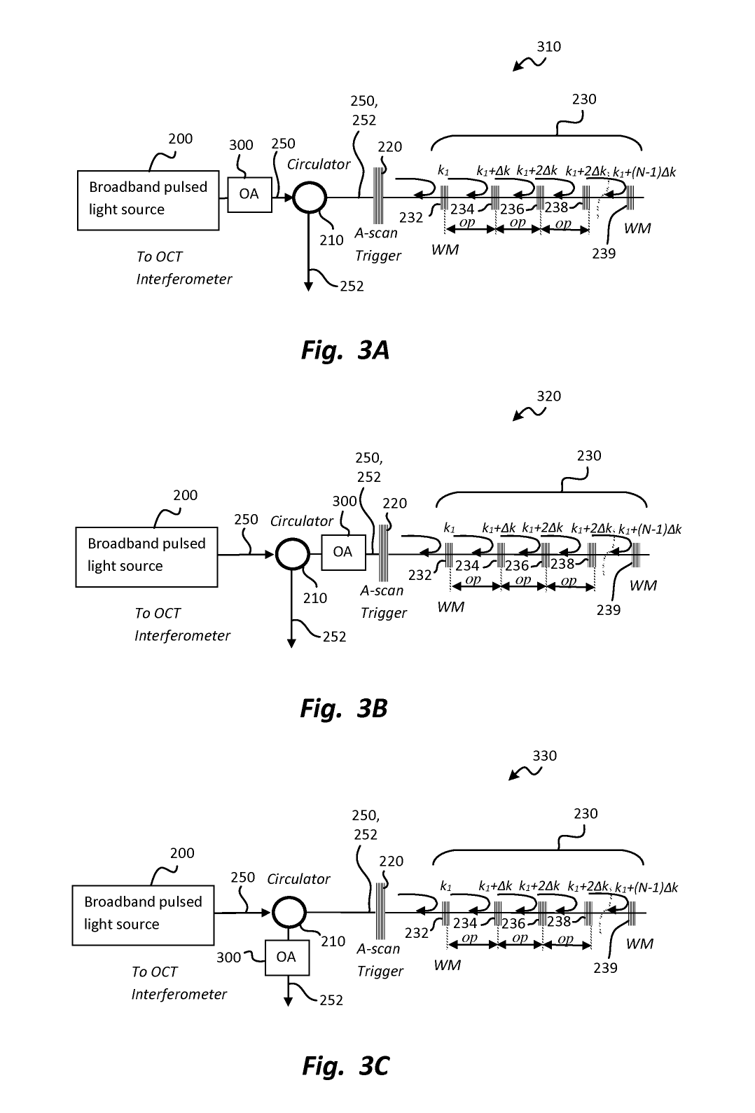

In the illumination component 120 of FIG. 2A and/or the illumination component 260 of FIG. 2B, losses may occur as light interacts with various components such as the reflectors. To maintain sufficient output power, an additional optical amplification of the light may be desirable. Various configurations including optical amplification will be shown and described in connection with FIGS. 3A, 3B, and 3C, as follows.

Referring to FIGS. 3A, 3B, and 3C, schematic diagrams illustrate three different illumination components that include optical amplification. The configurations of FIGS. 3A, 3B, and 3C may generally be based on that of FIG. 1A.

Specifically, an optical amplifier 300 (“OA”) can be placed between light source 200 and the circulator 210, as in the illumination component 310 of FIG. 3A. Thus, amplification may be carried out during forward direction of light propagation.

Additionally or alternatively, the optical amplifier 300 may be placed between the circulator 210 and the reflector system 230, as in the illumination component 320 of FIG. 3B. In this example, amplification may be carried out during both forward and/or reverse directions of light propagation.

Additionally or alternatively, the optical amplifier 300 may be placed between the circulator 210 and the remainder of the system, such as between the circulator 210 and the splitter 122, as in the illumination component 330 of FIG. 3C. Amplification may then be carried out during transmission of the light from the illumination component 330 to the remainder of the optical coherence tomography system.

A wide variety of optical amplifiers may be used, including but not limited to Semiconductor Optical Amplifiers (SOAs). Such SOA's may be standard optical elements purchased from any of a variety of companies, or may be custom-manufactured for use with an illumination component according to the present invention, such as the illumination component 310, the illumination component 320, and/or the illumination component 330.

With particular reference to FIGS. 2A, 3A, 3B, 3C, the wavelength groups may be evenly distributed spatially, due to the even optical pathway spacing increment (“op”) of FIGS. 2A, 3A, 3B, and 3C. Accordingly, the wave numbers (optical frequencies) may be equally distributed in time, i.e. dk/dt=constant. Thus, during equal time spans, the wavenumbers may change by the same value. To achieve linear dependence of wavenumber versus time (linear k-space sampling), the optical pathway length as well as wavenumber step between neighboring wavelength/wavenumber reflectors can be kept constant as shown in FIG. 2A. In general, to vary the wavenumber step between reflectors in proportion with the distance between reflectors, the following rule may be followed:

Δk(n)=α*Δop(n)

where n is the index of the reflector, Δk is the wavenumber, Δop the optical path length steps between adjacent wavenumber reflectors, and a is a constant. This equation may ensure that linear k-space sampling occurs, i.e. that dk/dt=const as mentioned above. In the case of a dispersive optical pathway when the refractive index depends on a wavelength relationship such as n=n(λ), either Δop(n) or Δk(n) or both can be spaced non-uniformly to keep the aforementioned rule true (i.e., maintain dk/dt=const). For regular single-mode fibers, a constant distance between reflectors and wavenumbers in most cases may not lead to detectable degradation of the OCT signal.

This may provide benefits over an approach such as a Fourier Domain OCT approach in which dk/dt≠const. Such an approach may require resampling to preserve the high resolution of the OCT. Resampling to the linear k-space may require additional processing time to process the OCT signal; this may complicate real-time A-scan data imaging. Moreover, additional information on the nonlinear distribution of the k-numbers versus time may be required for resampling.

In a swept source OCT approach, the non-linear sweeping rate in the k-space may lead to the need for an additional interferometer which may 1) complicate the system and consume useful optical power from the source, 2) use an additional ADC (analog-to-digital convertor) channel thus putting an extra requirements on the acquisition rate of the ADC (for example, requiring the ADC acquisition rate to be twice the OCT repetition rate), and/or 3) limit the imaging depth by the resample step in the k-space instead of the instantaneous swept source coherence length, thus potentially reducing the imaging depth of the OCT system. Therefore, with a distributed reflector illumination component such as the illumination component 120 and/or the illumination component 260 may simplify construction of the OCT system, reduce ADC requirements, and/or facilitate simultaneous real-time OCT signal acquisition and display.

Additional features of the invention will be discussed with continued reference to FIG. 1. Those of skill in the art will recognize that such features may be used in conjunction with other embodiments of the invention, including but not limited to the embodiments of FIGS. 1B, 2A, 2B, and 2C. Indeed, the features and embodiments described herein may be combined in a wide variety of ways that will be evident to a person of skill in the art with the aid of the present disclosure.

In some embodiments, the light source 200 may be of a type used in conventional optical coherence tomography. Alternatively, a different type of light source may be used. In some embodiments, the light source 200 may be a broadband pulsed light source in the form of a supercontinuum laser source. Exemplary devices are available from NKT Photonics, Inc. of Morganville, N.J.) or Fianium of Southampton in the United Kingdom.

Advantageously, a supercontinuum light source may be cheaper and/or more stable than a femtosecond lasers, and may provide an ultra-wide wavelength spectrum. By generating the broadband light using nonlinear fiber processes, a supercontinuum source may generate the majority of the light within a fiber, which may lead to very high spectral density. Thus, the amount of light within a single transverse mode per wavelength range may be very high compared to other sources. The supercontinuum light source may provide an ultra-broadband pulse with a pulse duration ranging from several picoseconds to hundreds of picoseconds; this duration may be well-suited for use with a distributed reflector system such as the reflector system 230 of FIG. 2A or the reflector system 280 of FIG. 2B.

For an OCT system such as the system 100 of FIG. 1, it may be desirable for the system 100 to recognize single A-scans, particularly if the illumination component used has a distributed reflector system such as the reflector system 230 of FIG. 2A or the reflector system 280 of FIG. 2B. An A-scan may correspond to a single depth profile. When spatially separated in an organized matter, a series of A-scans may be used to create cross-sectional B-scan images. A set of spatially separated B-scans may be used to create 3-D OCT images.

Each A-scan may be built up from a single pulse of the illumination component 120 through dispersal of the pulse via the distributed reflectors of the reflector system 230. Therefore, the output of the light source 200 may be used as an A-scan trigger. However, due to the ultra-fast repetition rate and finite speed of light, the A-scan trigger timing tolerance for the reflector system 230 in the system 100 may be very tight; the A-scan trigger pulse (i.e., a signal transmitted by the trigger 220) to the ADC may advantageously lead the corresponding A-scan data by a time interval ranging from a few nanoseconds to tens of picoseconds.

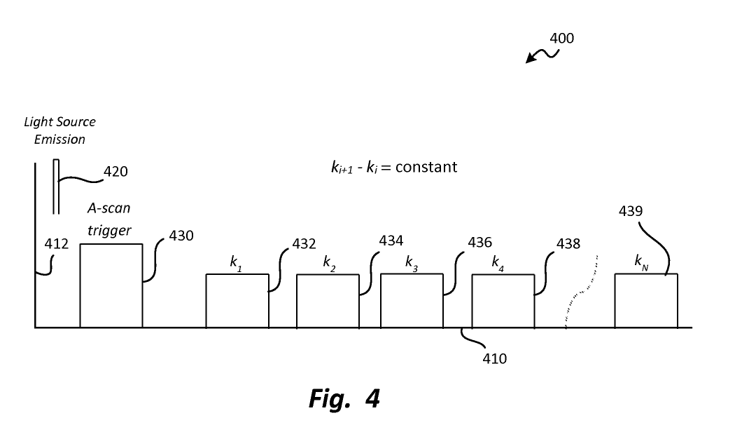

Referring to FIG. 4, a chart 400 illustrates the exemplary formation of optical output from an illumination component such as the illumination component 120. As shown, the chart 400 may have an X-axis 410 and a Y-axis 412. The X-axis 410 may represent time, and the Y-axis 412 may represent the intensity of light emitted by the illumination component 120.

A light source emission 420 may first be emitted by the light source 200 within the illumination component 120; this light may then interact with the A-scan trigger 220 and/or the reflector system 230 to trigger additional pulses. As shown, these may include an A-scan trigger pulse 430, a first reflected pulse 432 from the first reflector 232, a second reflected pulse 434 from the second reflector 234, a third reflected pulse 436 from the third reflector 236, a fourth reflected pulse 438 from the fourth reflector 238, and an nth reflected pulse 439 from the nth reflector 239.

As illustrated in FIG. 4, each adjacent pair of pulses of A-scan data, i.e., the first reflected pulse 432, the second reflected pulse 434, the third reflected pulse 436, and the fourth reflected pulse 438 may be separated from each other by the same time interval. This may be due to the constant optical pathway length differentiation between the first reflector 232, the second reflector 234, the third reflector 236, and the fourth reflector 238, as described previously. The A-scan trigger pulse 430 may lead the first reflected pulse 432, the second reflected pulse 434, the third reflected pulse 436, the fourth reflected pulse 438, and the nth reflected pulse 439.

In order to provide the necessary lead interval for the A-scan trigger (i.e., the time gap between the trigger pulse 430 and the first reflected pulse 432, as shown in FIG. 4), the length tolerance between the A-scan trigger and data travel distance (optical+electrical paths) may be in the range of 0.3-30 cm. This tolerance may be difficult to achieve if the A-scan trigger pulse and the A-scan data (i.e., the first reflected pulse 432 through the nth reflected pulse 439) travel along different pathways because matching the lengths of the pathways may be very difficult. In general, the A-scan trigger pulse and the light containing the A-scan data may both travel as much as tens of meters from the broadband pulsed light source to the ADC. This may make it difficult to obtain a pathway length differential on the order of a centimeter or less.

One way to facilitate the achievement of such tolerances may be the use of A-scan trigger and A-scan data pathways that are significantly overlapped. Since the optical and electrical pathways that do not overlap may be relatively short, the pathway length differential tolerances, as a percentage of non-overlapping pathway length, may be much larger than would be achieved without overlapping paths for the A-scan trigger pulse 430 and A-scan data.

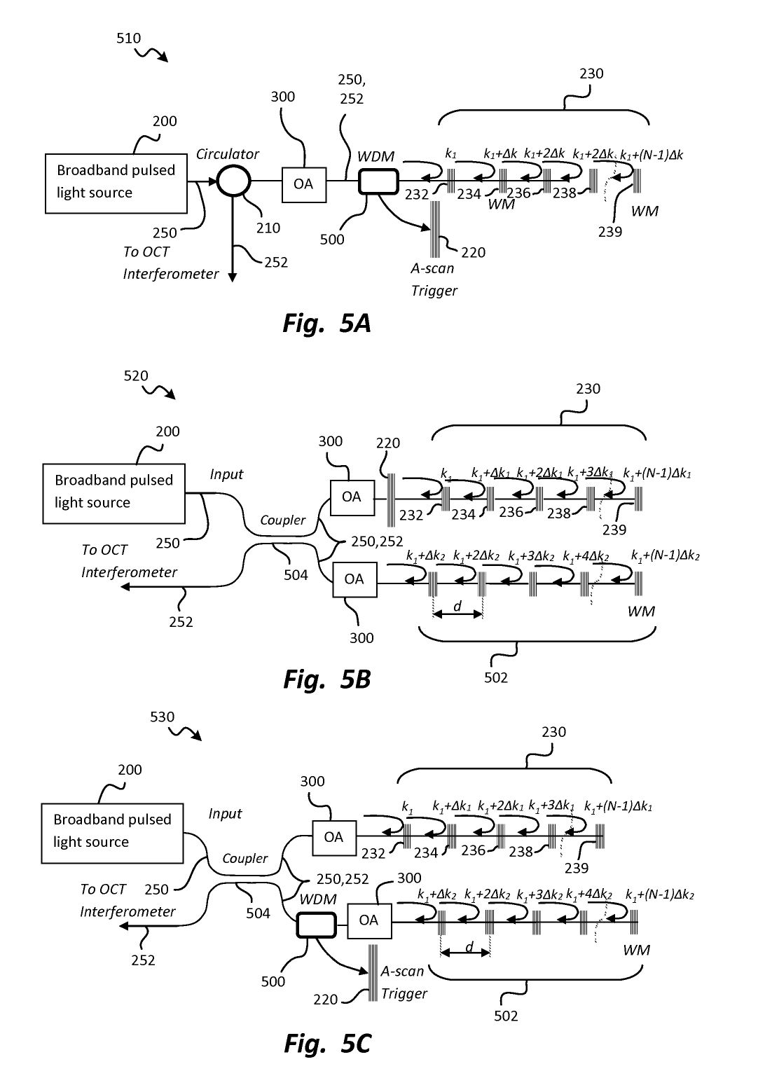

Referring to FIGS. 5A, 5B, and 5C, various illumination components illustrate exemplary schema for combining optical and/or data pathways for A-scan pulses and A-scan data. More precisely, FIG. 5A illustrates an illumination component 510 that utilizes a wavelength division multiplexer, or WDM 500, in connection with the trigger 220. FIGS. 5B and 5C illustrate an illumination component 520 and an illumination component 530, respectively, that utilize a second reflector system 502 that further subdivides the pulse from the light source 200 into wavelength groups. The second reflector system 502 may be configured in a manner similar to that of the reflector system 230. The broadband light 250 from the light source 200 may be divided between the reflector system 230 and the second reflector system 502 by a coupler 504. FIG. 5B may not utilize a WDM 500, while FIG. 5C may include a WDM 500.

As also shown in the FIGS. 2A, 3A, 3B, and 3C, the A-scan trigger 220 may be formed by introducing an additional reflector that is reflecting the wavelength band that is outside or inside the A-scan data wavelength band. The reflector that defines the A-scan trigger 220 may be placed before the reflector system 230 as in FIGS. 2A, 3A, 3B, 3C, 5A, and 5B and/or before the second reflector system 502 as in FIG. 5C. Additionally or alternatively, the A-scan trigger 220 may be separated from the light pathway followed by light entering the reflector system 230 and/or the second reflector system 502 by the WDM 500, as in FIGS. 5A and 5C.

The amplitude of the A-scan trigger pulse 430 may be made greater than the amplitudes of the A-scan data pulses such as the first reflected pulse 432, the second reflected pulse 434, the third reflected pulse 436, the fourth reflected pulse 438, and/or the nth reflected pulse 439 of FIG. 4. In such a case, the A-scan trigger pulse 430 may be differentiated by amplitude from the A-scan data.

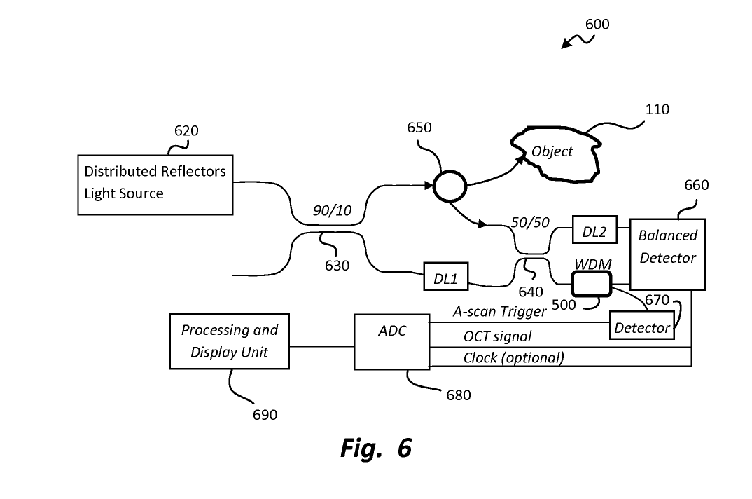

Referring to FIG. 6, a schematic view illustrates an optical coherence tomography system, or system 600, in which the A-scan trigger pulse 430 is separated from the A-scan data through the use of a WDM 500 in the detector pathway of the OCT system. The system 600 may be designed to image an object 110, and may include an illumination component 620, which may be like the illumination component 120 of such as that of FIG. 2A. Further, the system 600 may include a first coupler 630, a second coupler 640, a circulator 650, a balanced detector 660, a detector 670, an analog-to-digital converter, or ADC 680, and a processing and display unit 690. The ADC 680 and the processing and display unit 690 may be constituents of the signal processing componentry 128 of FIG. 1.

The various components of the system 600 may be connected as shown, such that a first delay DL1 and a second delay DL2 exist in the optical and/or data pathways of the system 600. The first delay DL1 may be offset from the second delay DL2 by a desired value. The trigger pulse 430 may be received by the detector 670, and the A-scan data (for example, the first reflected pulse 432 through the nth reflected pulse 439) may be received by the balanced detector 660. The trigger pulse 430, A-scan data (or OCT signal), and optionally, clock values, may be conveyed to the ADC 680, which may provide digital output to the processing and display unit 690.

The amplitude of the trigger pulse 430 can be any value accepted by the ADC. Additionally or alternatively, when separating the A-scan trigger pulse 430 from the A-scan data through the use of the WDM 500, any of the A-scan data pulses (e.g., the first reflected pulse 432 through the nth reflected pulse 439 of FIG. 4) can be used as a trigger by selectively branching part of the power at a specific wavenumber and adjusting the delay to the desired value by using a delay line either in the A-scan data pathway (optical and/or electrical) or the A-scan trigger pathway (optical and/or electrical).

In alternative embodiments (not shown), an intensity coupler may be used in the detector arm of the system 600 to form an A-scan trigger instead of the WDM 500 of FIG. 6 when the A-scan trigger pulse 430 has a higher amplitude than any of the A-scan data pulses. This may be achieved, for example, by using a light source 200 in the form of a supercontinuum or femtosecond laser source where the amplitude of the output pulse near the pumping wavelength is significantly higher than the amplitudes near any other wavelengths constituting the output pulse.

In other alternative embodiments, the OCT interferometer configuration for a distributed reflectors-based OCT can be different than that represented in FIG. 6. For example, a single channel detector (not shown in FIG. 6) can be used in place of the balanced detector 660 for A-scan data detection. However, a balanced OCT detection scheme as in FIG. 6 may provide higher optical signal-to-noise-ratio (OSNR) in the system 600 and may simplify data processing as compared with the single detector OCT system (not shown).

The ADC 680 of FIG. 6 may be internally clocked (not shown in the FIG. 6) when k-numbers in the A-scan data are varying linearly in time in accordance with the equation set forth above such that dk/dt=const. Additionally or alternatively, the ADC 680 may be externally clocked without the use of an additional interferometer due to the pulsative nature of the distributed A-scan data signals. Even when an A-scan data pulse train is in linear k-space, an external clock can be implemented to effectively use the ADC bandwidth and optical power, for example, by causing digitization events to take place mostly during times when A-scan data power is present.

The light before interference that passes through the coupler 640 may be beneficial to use for the optional clock, since some clock cycles can be too small due to the existence of distractive interference events at the specific wavelength value. For this purpose, the light upstream of the coupler 640 can be branched and detected by an additional single channel detector (not shown in FIG. 6) to create an optional clock. Additionally or alternatively, an additional interferometer (not shown in FIG. 6) may be used for external clocking of the ADC 680. One or more electrical circuits (not shown) may optionally between the ADC 680 and associated clock/trigger detectors, such as the balanced detector 660 and/or the detector 670 to adjust the signal shapes and/or values to those accepted by ADC 680.

With reference to the various embodiments disclosed herein involving an illumination component with distributed reflectors, such as the system 100 and/or the system 600, various system components may be available commercially. For the reflector system 230 and/or the second reflector system 502, a fiber Bragg grating (FBG) array may advantageously be used. For the ADC 680, an ultra-fast analog-to-digital converter such as a GHz range ADC card may be used. For the balanced detector 660 and/or the detector 670, an ultra-fast photodetector such as a GHz range photodetector may be used. For the light source 200, a broadband pulsed light source, such as a supercontinuum light source may be used.

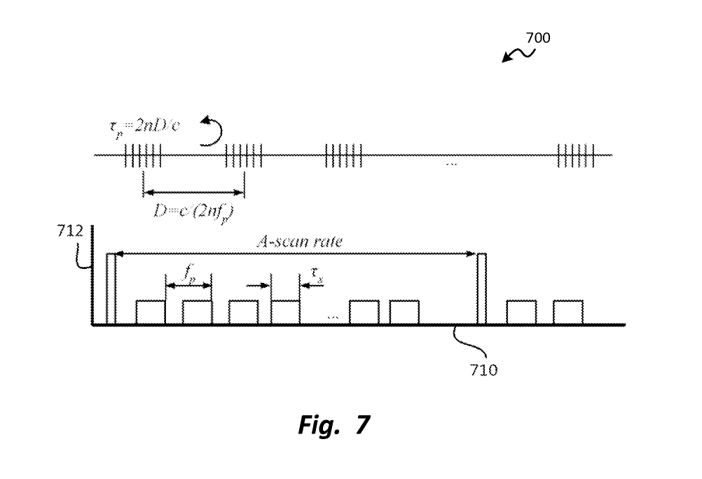

Referring to FIG. 7, a chart 700 illustrates the exemplary formation of optical output from an OCT system with commercially available components as set forth above. As shown, the chart 700 may have an X-axis 710 and a Y-axis 712. The X-axis 710 may represent time, and the Y-axis 712 may represent the intensity of light emitted by an illumination component such as the illumination component 120.

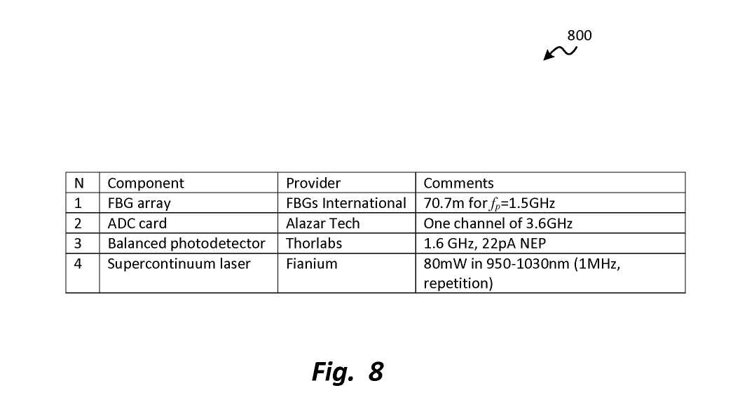

Referring to FIG. 8, a table 800 illustrates the parameters of major components that may be used to implement a distributed reflector OCT system as described above. The components, providers, and specifications listed are merely exemplary.

With reference to the exemplary data of FIG. 7 and FIG. 8, the A-scan acquisition speed of the OCT system may be limited either by the 1.8 GHz ADC card or the 1.6 GHz balanced photodetector bandwidth. A 1.5 GHz (fₚ) repetition rate of the pulses may correspond to a D=c/(2nfₚ)=6.9 cm distance between FBG gratings. Here, n, may be the fiber refractive index, and may be equal to 1.45. The coefficient 2 may be due to the double pass of the light between neighboring FBGs. Therefore the total length of the fiber (1024*D) may be 70.7 m. For the supercontinuum laser, the repetition rate may advantageously be slower than fₚ/1024, or 1.47 MHz (fₚ=1.5 GHz) to avoid overlapping A-scans. Notably, the A-scan rate may be equal to the repetition rate of the broadband pulsed light source 200. The spectrally limited pulse width τₛ may advantageously be shorter than the total period of the pulse propagation τₚ=1/fₚ to avoid overlapping pulse groups. For the case above, this may suggest a bandwidth of the single FBG of δλ=0.04 nm, τₛ=λ²/(cδλ)=83 ps<τₚ=700 ps.

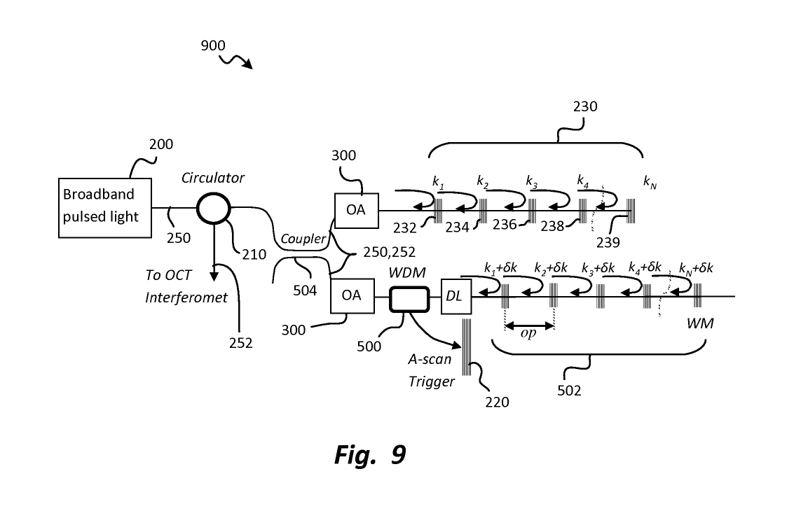

Referring to FIG. 9, a schematic view illustrates an illumination component 900 according to another alternative embodiment of the invention. As shown, the illumination component 900 may have several components in common with illumination components of previous embodiments, and particularly, with FIG. 5C. Thus, the illumination component 900 may have a light source 200, a circulator 210, an A-scan trigger 220, a reflector system 230, a second reflector system 502, and a WDM 500.

The second reflector system 502 may further subdivide the pulse from the light source 200 into wavelength groups. The second reflector system 502 may be configured in a manner similar to that of the reflector system 230. The broadband light 250 from the light source 200 may be divided between the reflector system 230 and the second reflector system 502 by the coupler 504. Optical amplifiers 300 may amplify the light prior to entry of the light into and/or exit of the light from the reflector system 230 and the second reflector system 502, as shown. The operation of the illumination component 900 may be similar to that of the illumination component 530 and other illumination components described herein.

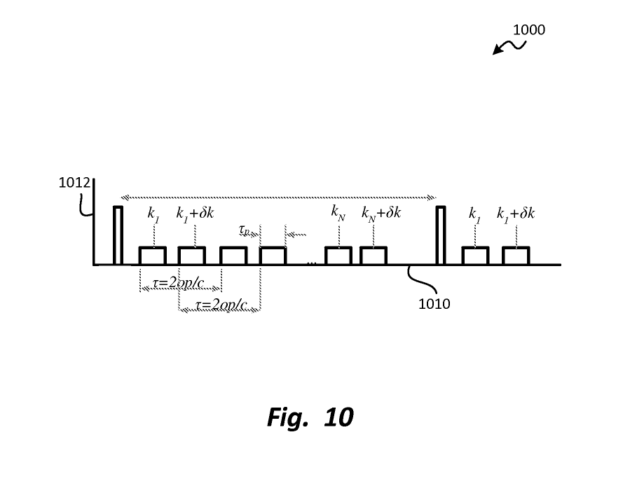

Referring to FIG. 10, a chart 1000 illustrates the exemplary formation of optical output from an OCT system including the illumination component 900 of FIG. 9, as described above. As shown, the chart 1000 may have an X-axis 1010 and a Y-axis 1012. The X-axis 1010 may represent time, and the Y-axis 1012 may represent the intensity of light emitted by the illumination component 900.

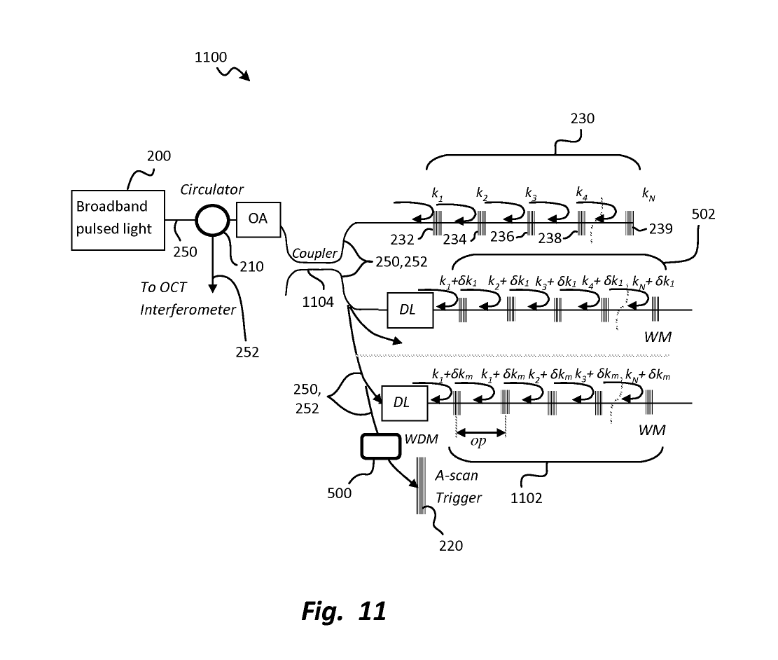

In an OCT system incorporating the illumination component 900 of FIG. 9 (not shown) and, by extension, other illumination components disclosed herein, the limiting factor of the A-scan rate may be the minimum distance required for separating adjacent k-number (wavelength) reflectors. For example, in one embodiment, the minimum distance between FBG gratings may be limited to, say 10 mm, so the gratings work independently and provide sufficient reflectivity. In such an embodiment, the A-scan rate may be limited to approximately 10.1 MHz for a 1024 elements distributed reflector and 5 MHz for a 2048 distributed reflector. To increase the A-scan rate above these levels, more than one reflector system may be used, as shown in FIGS. 5B, 5C, 9 and 11. FIGS. 5B, 5C, and 9 illustrate the use of two reflector systems and FIG. 11 illustrates any number of reflector systems, each of which may include a plurality of distributed reflectors.

As shown in FIGS. 5B, 5C, and 9, two such reflector systems may be shifted by Δk wavenumbers in their reflection spectral responses. It may be advantageous to set δk=0.5Δk (FIG. 9), where Δk is the wavenumber step between adjacent wavenumber reflectors for both reflectors. In general, δk may be any fraction of Δk. Note that it may be easy to avoid overlapping the data pulses from different reflector systems since the time duration of a single OCT data pulse (τₚ, FIG. 10) may be kept shorter than the time duration of required for a double light trip between two adjacent wavenumber reflectors (τ=2op/c, FIG. 10).

In the specific case of a Gaussian shaped wavenumber reflector, the bandwidth-limited pulse time duration can be derived from the Uncertainty principle:

τₚₗΔf=0.44

leading to τₚₗ=0.44λ²/(Δλc). Here, τₚₗ may be the time duration of a single OCT data pulse τₚ limited by a Gaussian spectrum with a width of Δf=Δλc/λ²; λ may be the central wavelength of the source, Δλ may be the bandwidth of the Gaussian wavenumber reflector, and c may be the speed of light. For example, given a wavelength λ of 1 μm, Δλ may be equal to 0.06 nm so that τₚₗ is equal to 25 ps. The 25 ps is shorter than τ=97 ps corresponding to a 10 mm distance between adjacent wavenumber reflectors. For non-Gaussian spectral reflection from wavenumber reflector, the equation set forth above may still be true with some variation in the value of the 0.44 coefficient, usually between 0.3 and 0.6. Therefore, one can infer that other overlapping estimations can be derived for the other spectral shapes that can be possessed by the reflections of wavenumber reflectors.

Referring to FIG. 11, a schematic view illustrates an illumination component 1100 according to another alternative embodiment of the invention. As shown, the illumination component 1100 may have several components in common with previously described embodiments. Thus, the illumination component 1100 may have a light source 200, a circulator 210, an A-scan trigger 220, a reflector system 230, a second reflector system 502, and a WDM 500. In addition to these components, the illumination component 1100 may have one or more additional reflector systems up to an nth reflector system 1102. The nth reflector system 1102 may be configured in a manner similar to that of the reflector system 230 and the second reflector system 502. The broadband light 250 from the light source 200 may be divided between the reflector system 230, the second reflector system 502, the nth reflector system 1102, and any other reflector systems included within the illumination component 1100 by a coupler 1104. The trigger 220 may be coupled to the remainder of the illumination component 1100 by a WDM 500.

In the illumination component 1100, the light from the light source 200 may be directed to the specific reflector system of the reflector system 230 through the nth reflector system 1102 by the corresponding branch of the coupler 1104. The wavenumber shift δkₘ may be chosen to construct a non-overlapping pulse train exiting the illumination component 1100. The wavenumbers and time distances between the pulses may advantageously be in correspondence with the first equation so that dk/dt=constant.

One example of a reflector system with distributed reflectors is the fiber Bragg grating (FBG) array. An FBG array of any number of elements can be built, for example, by Draw Tower Grating (DTG) technology whereby FBGs are written by UV laser-based interferometry during the fiber drawing process (FBGs International, Geel Belgium). Alternatively, FBGs can be written by UV lasers onto fibers with photosensitive cores and/or claddings. The material components of the fiber used for generating FBGs can possess higher losses than standard fibers. Therefore, to keep the output sample OCT power in the desired range (hundreds of μW, a few mW or tens of mW and higher), compensating fiber-based SOAs and/or other optical amplifiers may be used. Additionally, to provide a relatively constant reflection coefficient over the FBGs, it may be beneficial to compensate for the light loss with shorter fiber length. Likewise, the reflection amplitudes of the FBGs may be increased by increasing the FBG's distance from the fiber entrance.

On the other hand, imaging depth of an OCT system with such distributed reflectors may be reciprocal to the line width of the FBG reflection. The longer the FBG, the narrower the reflection line width can be made. Therefore, increasing the imaging depth may result in increasing the length of the fiber. Increasing the length of each of the FBGs can also be used to increase or control the power of a specific spectral component as well.

In another embodiment of the invention, an FBG may be continuously-chirped over the fiber length. The chirp can be made to satisfy the first equation above regarding a constant optical pathway increment between adjacent reflectors. In this case, the k-number step may satisfy the equation Δk=χcΔt/(2*n), and therefore the imaging depth (Δz˜2/Δk) may be determined by the ADC digitization time step Δt. Here, χ=dk/dl may be the rate of change in the wavenumber per fiber length, c is the speed of light in a vacuum, and n is the fiber refractive index. For OCT systems with distributed reflectors based on continuously chirped FBG, there may be no limitation on the A-scan repetition rate, as may be the case in an OCT system based on a set of distinct FBG's, each of which reflects a fixed wavenumber. Therefore, a continuously chirped FBG may be used for OCT systems with distributed reflectors operating at up to 80 MHz and faster.

Other limiting factors to increasing the acquisition rate of an OCT system with distributed reflectors (such as the system 100 of FIG. 1) may include the bandwidth of the ultra-fast balanced (single) photodetectors and the acquisition rates of the ADC digitizers, which may not be sufficient, but are approaching rates that keep pace with available broadband pulsed sources with repetition rates of ˜80 MHz and faster.

The acquisition rate of the photodetector and ADC may be approximately a laser repetition rate factor of the number of distributed reflectors. Thus, the acquisition rates for the photodetector and ADC may be approximately 40 GHz, 80 GHz, 160 GHz and 320 GHz for 512, 1024, 2048 and 4096 distributed reflectors, respectively. The number of distributed reflectors may be less than 512 or larger than 4096 and need not necessarily be an integer power of 2. However, use of a number of reflectors that is an integer power of two may increase the speed of Digital Fourier Transformations (DFT) by implementing a Fast Fourier Transformation (FFT) algorithm widely used in Fourier Domain OCT. Relatively rapid rates in photodetectors of 25 GHz and 40 GHz may be achieved by Alphalas (Goettingen, Germany) and by Discovery Semiconductors (Ewing, N.J., USA), respectively. The Alphalas photodetector uses a single detector, and the Discovery Semiconductors photodetector uses a balanced detector.

High-speed analog-to-digital converters suitable for the present invention may be obtained from a variety of sources. An analog-to-digital converter with a 12-bit ADC rate of 3.6 Gigasamples/s may be obtained from Texas Instruments (Dallas, Tex.), Alazar Tech (Pointe-Clair, QC Canada), and/or Ultraview Corporation (Berkeley, Calif.). An analog-to-digital converter with an 8-bit ADC of 56 Gigasamples/s may be obtained from Fujitsu Europe (Langen, Germany), and an analog-to-digital converter with a 6-bit ADC of 34 Gigasamples/s may be obtained from Micram (Bohum Germany and Burlington, Calif., USA).

Many current commercial and research OCT systems with acquisition rates of tens of thousands to a few hundred thousand A-scans/s are using 12-bit ADCs. OCT systems using 8-bit ADC's may have quality that is decreased, but still sufficient for some OCT imaging tasks. OCT systems using 14-bit ADC's may provide some increase in image quality over OCT systems with 12-bit ADC's, while an OCT system with a 16-bit ADC may not provide any advantage over those with 14-bit ADC's due to limitations posed by scattering photon noise within the dynamic range of the OCT signal. Rapid development and associated price reductions of the ultra-fast photodetectors and ADC's may facilitate the commercialization of OCT systems that utilize distributed reflectors to obtain A-scan acquisition rates of 80 MHz or faster.

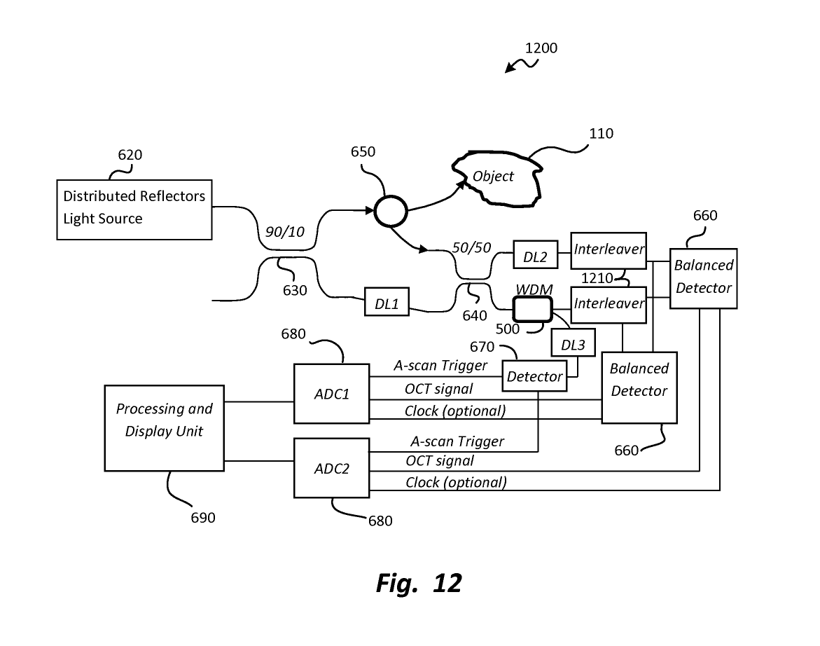

An increase in photodetector bandwidth may be accompanied by a reduction in its dynamic range; additionally, an increasing ADC digitization rate may lead to a decrease in the bit depth of the OCT image. Accordingly, the use of currently available ultra-high speed photodetectors and ADCs may lead to a decrease in the OCT signal-to-noise ratio and therefore, the quality of the OCT imaging. To increase the OCT acquisition rate while keeping the same photodetector and ADC acquisition rate, parallel signal acquisition of the OCT signal may be implemented, as will be shown and described in connection with FIG. 12.

Referring to FIG. 12, a schematic diagram illustrates an optical coherence tomography system, or system 1200, in which interleaving of the A-scan data is used to expedite analog-to-digital conversion. The system 1200 may be designed to image an object 110, and may include an illumination component 620, a first coupler 630, a second coupler 640, a circulator 650, two balanced detectors 660, a detector 670, two analog-to-digital converters, each of which is shown as an ADC 680, and a processing and display unit 690. The various components of the system 1200 may also be similar to their counterparts of the system 600 of FIG. 6.

The various components of the system 1200 may be connected as shown, such that a first delay DL1, a second delay DL2, and a third delay DL3 exist in the optical and/or data pathways of the system 1200. The first delay DL1, the second delay DL2, and the third delay DL3 may be offset from each other by a desired value. The trigger pulse 430 may be received by the detector 670, and the A-scan data (for example, the first reflected pulse 432 through the nth reflected pulse 439) may be received and divided by the interleavers 1210 so that the A-scan data is divided between the balanced detectors 660. The trigger pulse 430, A-scan data (or OCT signal), and optionally, clock values from each balanced detector 660 may be conveyed to one of the ADC's 680, which may provide digital output to the processing and display unit 690. The processing and display unit 690 may combine the output of the two ADC's 680 into one OCT image.

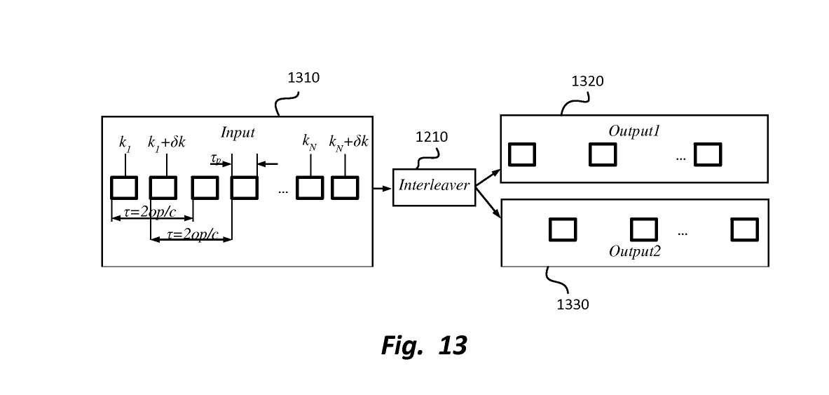

Referring to FIG. 13, a schematic view illustrates an interleaver 1210 as in FIG. 12. As shown, the interleaver 1210 may be designed to receive input 1310, and to divide the input 1310 into a first output 1320 and a second output 1330. In FIG. 12, the first output 1320 and the second output 1330 can be transmitted to separate balanced detectors 660, and the resulting data may be sent to separate ADC's 680 for independent processing.

The interleaver 1210 may be of a type known for use in the telecommunications industry, or may be custom-made for use in OCT systems according to the present invention. The interleaver 1210 may divide the input 1310 into the first output 1320 and the second output 1330 by, for example, splitting odd and even pulses of the input 1310 from a single optical channel into two different optical channels.

The system 1200 of FIG. 12 may utilize a balanced detection OCT setup, with two interleavers 1210 that split the responses from odd and even reflectors of the associated reflector systems (such as the reflector system 230) into outputs to the two balanced detectors 660 that direct electrical conversions of the optical signals into two ADC's 680. The ADC's 680 may direct the digital signal to the processing and display unit 690 where two signals can be combined again to construct the OCT signal and thence, the single OCT image. The output from the two balanced detectors 660 can be used for OCT data and k-space clock inputs of to the two ADC's 680 via two separate channels, as in FIG. 12, or the output from the balanced detectors 660 may instead be used for OCT data inputs into two distinct channels of a single ADC (not shown). Clock-designated single channel detectors can alternatively or additionally be used as well.

To simplify combining two or more data sets from the ADC's 680 back into one using the process and display unit 690, the order of the reflecting k-numbers in the wavenumber distributed reflectors may be optimized. In the exemplary case of two data streams entering the process and display unit 690, instead of using N equally-distributed k-numbers between kₛₜ and kₛₜ+N*Δk with the Δk step as shown in FIGS. 2A, 3A, 3B, 3C, 5A, 5B, and 5C above, the k-numbers of the odd reflectors may be evenly distributed between kₛₜ and kₛₜ+N/2*Δk with the Δk step, and the k-numbers of the even reflectors may be evenly distributed between kₛₜ+(N+2)/2*Δk and kₛₜ+N*Δk with the same step as illustrated in FIG. 13. In this case, the even data set may just be added at the end of the odd data set to provide a single data set ready for standard OCT processing. When the number of data sets entering the process and display unit 690 is increased, the subdivision of reflection wavenumber profiles may also be increased accordingly to simplify constructing a single data set. This may facilitate the repeated addition of an appropriate data set at the end of another data set.

In the system 1200 of FIG. 12, the requirements for the bandwidth of the balanced detectors 660 and the digitation rates of the ADC's 680 may be reduced by a factor of two, by comparison with an OCT system lacking an interleaver 1210, such as the system 600 of FIG. 6. The number of interleavers 1210 and the corresponding number of the balanced photodetectors 660 and ADC's 680 (or ADC channels) in an OCT system may be increased beyond the number used in the system 1200 of FIG. 12 to further decrease the requirements for the bandwidth of the balanced detectors 660 and the digitation rates of the ADC's 680. The interleavers 1210 may then be organized in a tree in which each interleaver 1210 in the tree has twice the Free Spectral Range (FSR) as the previous interleaver 1210.

The A-scan acquisition rate of an OCT system with distributed reflectors, as in several of the embodiments disclosed previously, may ultimately be limited by the pulse repetition rate of the broadband pulsed light source used in the illumination component of the OCT system. A pulse repetition rate of about 80 MHz may be achieved with known femtosecond and supercontinuum light sources suitable for OCT systems with distributed reflectors. As described above, the 80 MHz rate may be achieved through the use of commercially available components with parameters summarized in FIG. 8. A further increase in the A-scan acquisition rate of such an OCT system may optionally be achieved by increasing the broadband pulsed light source repetition rate through, for example, shortening of the laser cavity optical pathway length.

In addition to reducing motion artifacts and allowing OCT imaging to be used in additional situations, an ultrafast (i.e., tenth of MHz or greater) A-scan or single lateral point acquisition rate may provide other benefits. For example, an OCT system with distributed reflectors may be combined with one or more other ultrafast imaging systems that have similar data acquisition rates. If desired, such an OCT system may be aligned and/or synchronized to the data acquisition of the one or more other ultrafast imaging systems. Such other ultrafast imaging systems may utilize, but are not limited to, imaging techniques such as ultra-wide laser scanning imaging for simultaneous retinal or other imaging modalities. Such a combination may increase the informational value of both techniques. An ultra-wide retinal imaging system made by Optos, Inc. of Dunfermline, Scotland incorporates laser scanning at a 30 MHz rate; such an imaging system may advantageously be combined with an OCT system operating at a high A-scan acquisition rate. The one or more other ultrafast imaging systems may, if desired, utilize the same signal processing componentry as the OCT system, thereby facilitating combination and/or synchronization between the systems.

OCT systems with distributed reflectors, as described herein, may be ideal tools for phase-sensitive depth-resolved measurements. Standard phase-sensitive measurements in OCT systems with functional imaging may require phase stabilization between A-scans. Two main sources of phase instability in OCT imaging are: 1) difficult-to-predict variation of the A-scan starting wavenumber (δk) from one A-scan to the next, and 2) relative motion between the OCT probe and the object layer under investigation (δz). The phase variation between A-scans (δφ) may be represented as:

δφ=2(nzδk+knδz+kzδn)

where k is a wavenumber, z is distance between the OCT probe and the target point or range in the object under investigation (i.e., the object 110), n is the refractive index, and δ is a partial variation in the corresponding parameter. Multiplication by two in the equation may be due to double-passage of the probing light to and from the object under investigation.

Variation in phase over 2π radians (equivalent to half the central OCT source wavelength movement) may make it impossible to obtain any phase information due to the 2π phase ambiguity. Many imaging phase stabilization techniques (both in hardware and software) may be used in conjunction with standard OCT systems to extract functional information from the objects under investigation. Such stabilization efforts may complicate the commercial application of phase-resolved OCT imaging.

For OCT systems with distributed reflectors, as in the present invention, uncertainty in the A-scan starting k-number (wavenumber) may be eliminated automatically due to the fixed wavenumber of the spectral reflector of the A-scan trigger 220 used to generate the A-scan trigger pulse 430. The relative motion between the OCT probe and the object under investigation (i.e., the object 110) may be efficiently frozen due to the ultrafast A-scan acquisition rate of the OCT system with distributed reflectors.

In addition to depth-resolved phase measurements between A-scans, OCT systems with distributed reflectors may open a variety of ways to do intra-A-scan depth-resolved or non-depth-resolved phase measurements. This may be facilitated by the relatively highly-controlled, easily-predictable and extremely stable k-number of each OCT pixel constituting the A-scan. Therefore, phase changes that take place between A-scans constituting pixels or groups of pixels may be tracked. Tracking phase changes between A-scans may allow further freezing of motion artifacts and offer the ability to acquire functional and/or morphological maps simultaneously, which may otherwise present challenge when tracking phase changes between A-scans.

One potential advantage of OCT systems with distributed reflectors may be the ability to select any desired order of the k-number stream entering the photodetector and subsequent digitization and processing units. If desired, a non-uniform (or non-sequential) order of k-numbers may be used. One possible use for this functionality may be the ability to simplify the implementation of interleavers, like the interleavers 1210 of FIGS. 12 and 13, to increase the A-scan acquisition rate.

Another use of a non-uniform (or non-sequential) order of k-numbers of the distributed reflectors may be compressed OCT sensing. In compressed OCT sensing, the number of pixels per light source bandwidth may be below the Nyquist limit. Moreover, if the number of specific depth reflections or layers that are being investigated is known, the optimal wavenumber profile (not in prescribed order) consisting of acquisitions below the Nyquist limit may be determined. Therefore, the number of digitizations per A-scan may be decreased, leading to an increased acquisition rate, and subsequently facilitating subsequent steps such as real-time OCT processing and/or highlighting specific layers in the object under investigation.

The use of distributed reflectors with a k-number profile optimized for compressed imaging may be used alone or in combination with distributed reflectors with the number of pixels equal or greater than Nyquist limit. In case of the combined usage of compressed and full-length distributed reflectors sets, the set that is currently used for imaging can be switched through the use of an optical switcher. A variety of optical and fiber optic switchers are used in the telecommunications industry and can be purchased, for example, from Agiltron (Woburn, Mass.).

In combination with supercontinuum light sources that provide an ultra-broad band spectrum (for example, from 400 nm up to 2000 nm), the distributed reflectors of an OCT system can be used for ultra-high resolution submicron imaging. The bandwidth that is covered by the distributed reflectors of the reflector system (e.g., the reflector system 230) need not be limited; therefore, ultra-high resolution may be achieved by using a large bandwidth that is sampled using a small number of reflectors based on the compressed sampling theorem. For example, to achieve a 0.5 μm resolution and a 3 mm imaging range in tissue with a refractive index of 1.4, the reflector system with full-length distributed reflectors may have 450 nm covered by 12,000 reflectors centered at 850 nm.

In existing Fourier Domain OCT approaches (both Spectral Domain and Swept Source), it may be challenging to achieve more than 4,096 pixels per A-scan. It may be extremely challenging to build a 12,000 pixel photodetector array in the Spectral Domain OCT due to dimensional restrictions and the need for high-cost diffraction-limited, high numerical aperture optics. In Swept-Source OCT, the number of pixels usually does not exceed 2,048 due to the short instantaneous coherence length and limited ADC digitization rate.

However, in an OCT system with distributed reflectors, 12,000 reflectors may be written relatively easily using Fiber Bragg Gratings (FBGs). The fiber may not pose sharp limits to the number of FBGs in a single fiber, and may still have a relatively compact size due to the ability to wind around without a noticeable change in the optical properties of the FBG. To this end, FBGs are available from companies such as QPS Photonics for a wide spectrum of wavelengths, such as 650 nm-1500 nm. Such FBG's may be sufficient to cover all three of the most popular OCT ranges, which are near 850 nm, 1050 nm and 1310 nm.

Moreover, to achieve ultra-high resolution OCT, a reflector system with compressed distributed reflectors may be optimized by optimizing the order of the k-numbers of the reflectors. Such optimization may not be possible with conventional OCT systems such as diffraction grating-based and standard swept source OCT systems. In such systems, the k-number order may be monotonically changed in a fixed matter.

The claims are not limited to the specific implementations described above. Various modifications, changes and variations may be made in the arrangement, operation and details of the implementations described herein without departing from the scope of the claims.

What is claimed is:

1. An optical coherence tomography system comprising:
a light source that projects light;
a first reflector system positioned to receive the light and reflect a first plurality of light rays of the light, the first plurality of light rays comprising:
a first light ray emitted along a first optical pathway, wherein the first light ray falls substantially within a first wavelength range;
a second light ray emitted along a second optical pathway, wherein the second light ray falls generally within a second wavelength range that does not overlap with the first wavelength range; and
a third light ray emitted along a third optical pathway, wherein the third light ray falls generally within a third wavelength range that does not overlap with the first wavelength range and does not overlap with the second wavelength range;
wherein the first reflector system is positioned such that the first optical pathway, the second optical pathway, and the third optical pathway converge to define a first beam of light and the second optical pathway is longer than the first optical pathway by a first optical pathway differential and the third optical pathway is longer than the second optical pathway by a second optical pathway differential substantially equal to the first optical pathway differential;
optical componentry that directs a subset of the first beam of light at an object to be imaged;
a first detector that detects the subset after reflection of the subset from the object to provide signals; and
signal processing componentry that processes the signals to provide an optical coherence tomography image of the object.

2. The optical coherence tomography system of claim 1, the second optical pathway is offset from the first optical pathway and the third optical pathway is offset from the second optical pathway and the third optical pathway, wherein the first reflector system comprises:
a first reflector positioned in the first optical pathway to reflect light to the first light ray;
a second reflector positioned in the second optical pathway to reflect light to the second light ray; and
a third reflector positioned in the third optical pathway to reflect light to the third light ray;
wherein the first, second, and third reflectors are offset from each other to provide the first optical pathway differential and the second optical pathway differential.

3. The optical coherence tomography system of claim 1, wherein the first, second, and third optical pathways are substantially collinear with each other, wherein the first reflector system comprises:
a first reflector positioned in the first optical pathway to reflect light to the first light ray;
a second reflector positioned in the second optical pathway to reflect light to the second light ray; and
a third reflector positioned in the third optical pathway to reflect light to the third light ray;
wherein the first, second, and third reflectors are offset from each other to provide the first optical pathway differential and the second optical pathway differential.

4. The optical coherence tomography system of claim 1, further comprising at least one selection from the group consisting of:
an optical amplifier positioned to amplify the light projected by the light source prior to division of the light into the first plurality of light rays;
an optical amplifier positioned to amplify the light of the first plurality of light rays; and
an optical amplifier positioned to amplify the first beam of light.

5. The optical coherence tomography system of claim 1, wherein the light source comprises a pulsed broadband supercontinuum laser.

6. The optical coherence tomography system of claim 5, wherein the light projected by the pulsed broadband supercontinuum laser has an ultra-wide bandwidth selected to facilitate ultra-high resolution optical coherence tomography imaging.

7. The optical coherence tomography system of claim 1, wherein the optical componentry further directs the subset of the first beam of light along a first beam pathway, the optical coherence tomography system further comprising an A-scan trigger configured to emit an A-scan trigger pulse along the first beam pathway to initiate detection of the subset by the first detector, wherein the A-scan trigger pulse is differentiable from the first beam via an amplitude differential between the A-scan trigger pulse and the first beam.

8. The optical coherence tomography system of claim 1, wherein the optical componentry further directs the subset of the first beam of light along a first beam pathway, the optical coherence tomography system further comprising:
an A-scan trigger configured to emit an A-scan trigger pulse; and
a wavelength division multiplexer that multiplexes the A-scan trigger pulse into the first beam pathway to initiate detection of the subset by the first detector;
wherein the A-scan trigger pulse is differentiable from the first beam via a wavelength differential between the A-scan trigger pulse and the first beam.

9. The optical coherence tomography system of claim 1, wherein inclusion of the first light ray, the second light ray, and the third light ray in the first beam of light induces wavelength-varied pulsing of the first beam of light, wherein the signal processing componentry comprises an analog-to-digital converter clocked by a selection from the group consisting of the wavelength-varied pulsing or a sine wave electronically produced from the wavelength-varied pulsing.

10. The optical coherence tomography system of claim 1, further comprising:
a second reflector system; and
a coupler that divides the light from the light source into a first portion received by the first reflector system and a second portion received by the second reflector system;
wherein the second reflector system reflects a second plurality of light rays of the second portion of the light, wherein the second plurality of light rays fall generally within wavelength ranges that are different from each other.

11. The optical coherence tomography system of claim 1, further comprising:
an interleaver that receives the first beam of light and divides the first beam of light into a first portion of the first beam and a second portion of the first beam, wherein the subset of the first beam detected by the first detector comprises the first portion of the first beam; and
a second detector that detects the second portion of the first beam.

12. The optical coherence tomography system of claim 1, wherein the first reflector system comprises a fiber Bragg grating array comprising fiber having a length, wherein the fiber has been substantially continuously chirped along a portion of the length.

13. The optical coherence tomography system of claim 1, wherein the signal processing componentry further processes other signals from one or more other ultrafast imaging systems different from the optical coherence tomography system.

14. The optical coherence tomography system of claim 1, wherein the signals comprise a plurality of A-scans, wherein the signal processing componentry is configured to process the A-scans independently of application of any phase stabilization technique.

15. The optical coherence tomography system of claim 1, wherein the first light ray comprises a first k-number, the second light ray comprises a second k-number, and the third light ray comprises a third k-number, wherein the first k-number, the second k-number, and the third k-number are non-sequential relative to an order in which the first light ray, the second light ray, and the third light ray are detected and are further selected to facilitate a process selected from the group consisting of:
interleaving the first beam of light to divide the first beam of light into a first portion comprising the subset detected by the first detector, and a second portion detected by a second detector; and
performing compressed optical coherence tomography sensing.

16. The optical coherence tomography system of claim 1, wherein the first reflector system comprises a fiber Bragg grating array comprising a plurality of reflectors, wherein the plurality of reflectors comprising a number of reflectors selected to facilitate a process selected from the group consisting of:
ultra-high resolution optical coherence tomography imaging; and
ultra-deep optical coherence tomography imaging.

17. An optical coherence tomography system comprising:
a light source that projects light;
a dispersive element that receives the light and divides the light into a plurality of light rays comprising:
a first light ray that travels along a first optical pathway;
a second light ray that travels along a second optical pathway offset from the first optical pathway; and
a third light ray that travels along a third optical pathway offset from the first optical pathway and the second optical pathway; and
a reflector system positioned to reflect the plurality of light rays such that the first light ray, the second light ray, and the third light ray converge to define first beam of light;
optical componentry that directs a subset of the first beam of light at an object to be imaged;
a detector that detects the subset after reflection of the subset from the object to provide signals; and
signal processing componentry that processes the signals to provide an optical coherence tomography image of the object.

18. The optical coherence tomography system of claim 17, wherein the dispersive element is configured such that:
the first light ray falls substantially within a first wavelength range;
the second light ray falls substantially within a second wavelength range that does not overlap with the first wavelength range; and
the third light ray falls substantially within a third wavelength range that does not overlap with the first wavelength range and does not overlap with the second wavelength range.

19. The optical coherence tomography system of claim 18, wherein the reflector system comprises a first reflector positioned in the first optical pathway to reflect the first light ray, a second reflector positioned in the second optical pathway to reflect the second light ray, and a third reflector positioned in the third optical pathway to reflect the third light ray, wherein the first, second, and third reflectors are offset from each other such that the second optical pathway is longer than the first optical pathway and the third optical pathway is longer than the second optical pathway such that, in the first beam of light, light within the second wavelength range lags behind light within the first wavelength range and light within the third wavelength range lags behind light within the second wavelength range.

20. An optical coherence tomography system comprising:
a light source that projects light;
a first reflector system positioned to receive the light and reflect a first plurality of light rays of the light, the first plurality of light rays comprising:
a first light ray emitted along a first optical pathway, wherein the first light ray falls substantially within a first wavelength range;
a second light ray emitted along a second optical pathway, wherein the second light ray falls generally within a second wavelength range that does not overlap with the first wavelength range; and
a third light ray emitted along a third optical pathway, wherein the third light ray falls generally within a third wavelength range that does not overlap with the first wavelength range and does not overlap with the second wavelength range;
wherein the first reflector system is positioned such that the first optical pathway, the second optical pathway, and the third optical pathway converge to define a first beam of light;
optical componentry that directs a subset of the first beam of light at an object to be imaged;
an interleaver that receives the first beam of light and divides the first beam of light into a first portion of the first beam and a second portion of the first beam;
a first detector that detects the first portion of the first beam to provide a first set of signals;
a second detector that detects the second portion of the first beam to provide a second set of signals; and
signal processing componentry that processes the first set of signals and the second set of signals to provide an optical coherence tomography image of the object.
